(19) United States
(12) Patent Application Publication
(10) Pub. No.: US 2026/0317712 A1
(43) Pub. Date: Oct. 15, 2026

(54) METHOD FOR CRYOPRESERVATION AND CULTURE OF DENDRITIC CELLS DERIVED FROM LYMPHOID TISSUES, AND COMPOSITION THEREFOR

(71) Applicants: NANOFAENTECH CO., LTD., Daegu (KR); AJOU UNIVERSITY INDUSTRY-ACADEMIC COOPERATION FOUNDATION, Gyeonggi-do (KR)

(72) Inventors: JONG YOUNG KWAK, Gyeonggi-do (KR); HUE VY AN TRAN, Gyeonggi-do (KR); THI XUAN YHUY TRAN, Gyeonggi-do (KR); IN JEONG LEE, Gyeonggi-do (KR); HYE YOUNG JEON, Gyeonggi-do (KR)

(21) Appl. No.: 18/871,114

(22) PCT Filed: Dec. 8, 2023
(86) PCT No.: PCT/KR2023/020192

§ 371 Date: Dec. 2, 2024

(30) Foreign Application Priority Data
Dec. 6, 2023 (KR) 10-2023-0175921

(51) Int. Cl. C12N 5/0784; C12N 5/00

(52) U.S. Cl. CPC C12N 5/0639; C12N 5/525; C12N 5/526; C12N 2501/998; C12N 2502/1323; C12N 2533/40

(57) ABSTRACT

A composition for cryopreservation of primary DCs includes serum and DMSO, such that it may have high viability after thawing the primary DCs and inhibited spontaneous activation to allow ex vivo culture, and may be utilized in immunological diagnosis of various diseases and development of therapeutics, as well as methods for freezing and storage, and cell culture using the same.

### BACKGROUND

### 1. Technical Field

The present invention relates to a method for cryopreservation and culture of primary dendritic cells isolated from lymphoid tissues.

### 2. Background Art

Dendritic cells (DCs) are antigen presentation cells to present the antigen to T cells, and are involved in association between innate immunity response and adaptive immunity response. The DCs are distributed in mucosa, intestine, and epidermal tissues as well as immune tissues such as spleen and lymph nodes, etc., and are present in the form of different subtypes. The subtypes of DCs are cells divided from those of macrophages or monocytes in the development stage and characterized in the terms of immunological role by presentation of antigens.

In a state of having no stimulation such as inflammation, the DCs are present in an inactive form and the inactive DCs have a low expression level of major histocompatibility complex class-II (MHC-II) surface molecules, show very little expression of costimulatory molecules, and rarely form inflammation-inducing cytokine. DCs having phagocytosis of external pathogens or antigens of damaged cells are activated and the activated DCs show decreased antigen absorption performance, increased MHC-II expression, increased expression of costimulatory molecules (CD80, CD86 and CD40), secretion of inflammation-inducing (pro-inflammatory) cytokine, and increased migration to lymph nodes. Such activated DCs may have antigen presentation function to induce T cell reaction to specific antigens.

The most significant problem in measuring the immunological function of stable DCs is involved in the isolation and culture of tissue-derived primary DCs. The primary DCs isolated for cell culture undergo spontaneous activation from inactive form to active form within 12 hours while being incubated. The spontaneously activated DCs cannot be incubated for a long period of time due to very short life. An extracellular matrix regulates interaction of immune cells and plays an important role in coagulation of cells in the tissue. Fibronectin as a major extracellular matrix is expressed in a lymphoid tissue. Unlike living tissues, it has been proposed that spontaneous activation in vitro culture occurs due to combination or contact between DCs under culture conditions. Further, it has been known that such spontaneous activation may be efficiently prevented by addition of fibronectin as a component of the extracellular matrix.

### SUMMARY

It is an object of the present invention to provide a composition for cryopreservation of primary dendritic cells (DCs) and tissues including the same, as well as a method for freezing and storage using the same.

Another object of the present invention is to provide a method for culturing primary DCs using the composition for cryopreservation and the method for freezing and storage.
1. A method for culturing primary dendritic cells (DCs), the method including freezing and storage of the primary DCs or tissues including the same in a composition including serum, dimethyl sulfoxide (DMSO) and fibronectin.
2. A method for culturing primary dendritic cells (DCs), the method including freezing and storage of the primary DCs or tissues including the same in a composition including serum, DMSO and albumin.
3. The method according to the above 1 or 2, further including thawing the primary DCs or tissues including the same, and then, culturing the cells or tissues in a medium including fibronectin.
4. The method according to the above 1 or 2, further including thawing the primary DCs or tissues including the same, and then, culturing the cells or tissues in a medium that does not include fibronectin.
5. The method according to the above 1 or 2, further including thawing the primary DCs or tissues including the same, and then, co-culturing the cells or tissues together with fibroblasts.
6. The method according to the above 1 or 2, further including: thawing the primary DCs or tissues including the same, and then, dispensing the primary DCs on a first scaffold which includes polyvinyl alcohol (PVA) nanofiber or polycaprolactone (PCL) nanofiber; and dispensing fibroblasts on a second scaffold which includes poly caprolactone (PCL) nanofiber thus to co-culture the primary DCs and the fibroblasts.
7. The method according to the above 6, wherein the first scaffold and the second scaffold are laminated in an incubator while being spaced apart from each other.
8. The method according to the above 2, wherein the composition further includes fibronectin.
9. The method according to the above lor 2, wherein the serum is 10 to 90% FBS.
10. The method according to the above 1 or 2, wherein the DMSO is 5 to 15% DMSO.
11. The method according to the above 1 or 8, wherein the fibronectin is included in an amount of 100 to 300 μg/mL.
12. The method according to the above 2, wherein the albumin is included in an amount of 300 to 500 mg/mL.
13. A composition for cryopreservation of primary DCs or tissues including the same, the composition including serum, DMSO and fibronectin.
14. A composition for cryopreservation of primary DCs or tissues including the same, the composition including serum, DMSO and albumin.
15. The composition according to the above 13 or 14, wherein the serum is 10 to 90% FBS.
16. The composition according to the above 13 or 14, wherein the DMSO is 5 to 15% DMSO.
17. The composition according to the above 13, wherein the fibronectin is included in an amount of 100 to 300 μg/mL.
18. The composition according to the above 14, wherein the albumin is included in an amount of 300 to 500 mg/mL.

The composition for cryopreservation and the method for freezing and storage according to the present invention may allow freezing and storage of primary DCs isolated from the spleen as body immune tissues.

The composition for cryopreservation and the method for freezing and storage according to the present invention may increase cell survival in a thawed state after cryopreservation of the primary DCs.

The composition for cryopreservation and the method for freezing and storage according to the present invention may inhibit spontaneous activation of the primary DCs in the thawed state after cryopreservation of the primary DCs.

The composition for cryopreservation and the method for freezing and storage according to the present invention may prevent spontaneous activation of the primary DCs even in a condition without fibronectin (ex vivo environment), in the thawed state after cryopreservation of the primary DCs.

The composition for cryopreservation and the method for freezing and storage according to the present invention may enable ex vivo culture of primary DCs.

The composition for cryopreservation and the method for freezing and storage according to the present invention may implement immunological response of primary DCs in a culture state.

The composition for cryopreservation and the method for freezing and storage according to the present invention may conduct co-culture of cryopreserved primary DCs together with T cells so that the primary DCs induce T cell proliferation and T cell cytokine secretion in the culture state.

The composition for cryopreservation and the method for freezing and storage according to the present invention may use primary DCs and contribute to measurement of immune response against various diseases (cancer, viral infection diseases, autoimmune diseases, inflammatory diseases, organ transplantation, allergy and rheumarthritis, etc.), immunological diagnosis, and development of immunological therapeutics.

### BRIEF DESCRIPTION OF THE DRAWINGS

FIG. 1 illustrates results of flow cytometry that measured a viability of spleen DCs (sDCs), which were obtained by adding fetal bovine serum (FBS) by concentrations in a state of adding 10% DMSO and 10% glycerol to sDCs, followed by freezing and storage for 7 days and then thawing the same.

FIG. 2 illustrates results of flow cytometry of sDCs after thawing, which were obtained by cryopreservation to sDCs in a solution added with 10% DMSO and 80% FBS and then thawing the same, wherein these are results of measuring the viability of freshly isolated sDCs, which are CD11c⁺ immediately after isolation thereof from the spleen, as well as CD86 expression level on the surface of cells.

FIG. 3 illustrates results of flow cytometry that measured the viability of sDCs, which were obtained by freezing and storing sDCs for 7 days in a composition for cryopreservation including bovine serum albumin (BSA) added by concentrations in a culture solution containing 10% DMSO and RPM1-1640, followed by thawing the same.

FIG. 4 illustrates results of flow cytometry that measured the viability of sDCs without cryopreservation, as well as CD11c and CD86 expression level on the surface of cells, wherein sDCs were isolated by adding 200 μg/mL of fibronectin and culturing the same for 24 hours in Example 3.

FIGS. 5A, 5B and 6 illustrate results of measuring the viability of sDCs thawed after cryopreservation in Example 4.

FIGS. 7 to 10 illustrate results of measuring the viability of sDCs thawed after cryopreservation in Example 5.

FIG. 11 illustrates scanning electron microscopic photographs of poly caprolactone (PCL) nanofibers and polyvinyl alcohol (PVA) nanofibers for co-culture of sDCs and fibroblasts.

FIG. 12 illustrates one embodiment of a co-culture system for sDCs and fibroblasts.

FIG. 13 illustrates results of measuring the viability of sDCs co-cultured with fibroblasts in Example 6.

FIG. 14 illustrates results of measuring the viability of post-culture cells of DCs isolated from the cryopreserved entire spleen cells in Example 7.

FIG. 15 illustrates results of measuring the cell viability and CD86 expression of DCs, wherein DCs freezing-stored in a composition containing 10% DMSO, 80% FBS, and 200 μg/mL of fibronectin were thawed, followed by removing dead cells, and the treated DCs were dispensed on PCL nanofiber membrane (PCL NM) and PVA nanofiber membrane (PVA NM) and then cultured together with 3 T3 cells for 24 hours.

### DETAILED DESCRIPTION

The present invention provides a composition for cryopreservation of primary DCs, and methods for freezing and storage, and cell culture using the same.

The present invention provides a composition for cryopreservation of primary DCs, including serum, DMSO and fibronectin, such that it may have high viability after thawing the primary DCs and inhibited spontaneous activation to allow ex vivo culture, and may be utilized in immunological diagnosis and development of therapeutics, as well as methods for freezing and storage, and cell culture using the same.

The composition for cryopreservation of the present invention may include serum, DMSO and fibronectin.

The serum is a component of blood, and may refer to a yellow liquid component remaining after removing fibrinogen from the serum. The serum includes water, glucose, inorganic salts, gamma-globulin or the like. The serum may include protein, electrolyte, antibody, antigen, hormone, and the like, while not including white blood cells, red blood cells, platelet and coagulation factors.

In one embodiment, the serum is fetal bovine serum (FBS). The FBS may include growth factor, protein, trace elements, vitamin, hormone, etc., which functions to preserve and maintain cells.

In one embodiment, the serum may include FCS, human AB serum, etc.

The serum may be included at a concentration of 10 to 90% (v/v). For example, the serum may be included in an amount of 10%, 15%, 20%, 25%, 30%, 35%, 40%, 45%, 50%, 55%, 60%, 65%, 70%, 75%, 80%, 85% or 90%.

The serum may be included in the amount of 100 μl, 150 μl, 200 μl, 250 μl, 300 μl, 350 μl, 400 μl, 450 μl, 500 μl, 550 μl, 600 μl, 650 μl, 700 μl, 750 μl, 800 μl, 850 μl or 900 μl, based on 1 mL of the composition for cryopreservation.

The DMSO refers to dimethyl sulfoxide. The DMSO may play a role of reducing cell damage due to ice crystals at the freezing and storage of cells.

The DMSO may be included at a concentration of 5 to 15% (v/v). For example, the DMSO may be included at a concentration of 5%, 6%, 7%, 8%, 9%, 10%, 11%, 12%, 13%, 14% or 15%.

The DMSO may be included in an amount of 50 μl, 60 μl, 70 μl, 80 μl, 90 μl, 100 μl, 110 μl, 120 μl, 130 μl, 140 μl or 150 μl, based on 1 mL of the composition for cryopreservation.

The fibronectin is a glycoprotein of extracellular matrix, and plays a role of inhibiting spontaneous activation of the primary DCs.

The fibronectin may be included at a concentration of 100 to 300 μg/mL. For example, the fibronectin may be included at a concentration of 100 μg/mL, 110 μg/mL, 120 μg/mL, 130 μg/mL, 140 μg/mL, 150 μg/mL, 160 μg/mL, 170 μg/mL, 180 μg/mL, 190 μg/mL, 200 μg/mL, 210 μg/mL, 220 μg/mL, 230 μg/mL, 240 μg/mL, 250 μg/mL, 260 μg/mL, 270 μg/mL, 280 μg/mL, 290 μg/mL or 300 μg/mL.

In one embodiment, a fibronectin stock prepared by dissolving 1 to 3 mg of fibronectin in 1 mL of medium may be used.

The fibronectin may be included at a concentration of 5 to 15% (v/v) in the composition for cryopreservation. The fibronectin may be included at a concentration of 5%, 6%, 7%, 8%, 9%, 10%, 11%, 12%, 13%, 14% or 15%.

The fibronectin may be included in an amount of 50 μl, 60 μl, 70 μl, 80 μl, 90 μl, 100 μl, 110 μl, 120 μl, 130 μl, 140 μl or 150 μl, based on 1 mL of the composition for cryopreservation.

The composition for cryopreservation of the present invention may further include serum albumin.

The serum albumin may be included in an amount of 300 to 500 mg/mL. For example, the serum albumin may be included in the amount of 300 mg/mL, 310 mg/mL, 320 mg/mL, 330 mg/mL, 340 mg/mL, 350 mg/mL, 360 mg/mL, 370 mg/mL, 380 mg/mL, 390 mg/mL, 400 mg/mL, 410 mg/mL, 420 mg/mL, 430 mg/mL, 440 mg/mL, 450 mg/mL, 460 mg/mL, 470 mg/mL, 480 mg/mL, 490 mg/mL or 500 mg/mL.

The composition for cryopreservation of the present invention may further include a medium required for storing and maintaining the primary DCs. Components and types of the medium are not particularly limited.

The composition for cryopreservation of the present invention may further include a component for inhibiting spontaneous activation of the primary DCs.

In one embodiment, the composition for cryopreservation may include 10% DMSO, 80% FBS, 10% of medium and cell pellets. Based on 1 mL of the composition for cryopreservation, it may include 100 μl DMSO (v/v), 800 μl FBS (v/v), 100 μl media and cell pellets.

In one embodiment, the composition for cryopreservation may include 10% DMSO, 80% FBS, 200 μg/mL of fibronectin and cell pellets. Based on 1 mL of the composition for cryopreservation, it may include 100 μl DMSO (v/v), 800 μl FBS (v/v), 100 μl fibronectin (stock 2 mg/mL) and cell pellets.

In one embodiment, the composition for cryopreservation may include 10% DMSO, 60% FBS, 30% of medium and cell pellets. Based on 1 mL of the composition for cryopreservation, it may include 100 μl DMSO (v/v), 600 μl FBS (v/v), 300 μl media and cell pellets.

In one embodiment, the composition for cryopreservation may include 10% DMSO, 60% FBS, 200 μg/mL of fibronectin, 20% of medium and cell pellets. Based on 1 mL of the composition for cryopreservation, it may include 100 μl DMSO (v/v), 600 μl FBS (v/v), 100 μl fibronectin (stock 2 mg/mL), 200 μl of medium and cell pellets.

The present invention provides a method for freezing and storage of primary DCs, which includes: freezing and storage of the primary DCs or tissues (spleen, etc.) including the same while immersing them in the composition for cryopreservation; then, thawing the primary DCs or tissues including the same; and culturing the thawed cells or cells obtained from the thawed cells.

The present invention provides a method for ex vivo culture of primary DCs, which includes: freezing and storage of the primary DCs or tissues including the same (spleen, etc.) while immersing them in the composition for cryopreservation; then, thawing the primary DCs or tissues including the same; and culturing the thawed cells or cells obtained from the thawed cells.

The primary DCs or tissues including the same may be cultured in a medium including fibronectin after thawing the same. The fibronectin may inhibit spontaneous activation of the primary DCs.

The primary DCs cells or tissues including the same may be cultured in a medium that does not include fibronectin after thawing the same. When using the composition for cryopreservation of the present invention, spontaneous activation of the primary DCs may be inhibited even if the cells are cultured in the medium that does not include fibronectin.

The primary DCs or tissues including the same may be co-cultured together with fibroblasts after thawing the same. If the cells are co-cultured with the fibroblasts, these may share useful substances such as cytokine and be helpful in cell growth.

The primary DCs and the fibroblasts may be co-cultured by thawing the primary DCs or tissues including the same, and then, dispensing the primary DCs on a first scaffold which includes polyvinyl alcohol (PVA) nanofiber or polycaprolactone (PCL) nanofiber; and dispensing fibroblasts on a second scaffold which includes poly caprolactone (PCL) nanofiber.

The first scaffold and the second scaffold may be laminated in an incubator while being spaced apart from each other. Two cells are present with physically apart from each other and may be indirectly co-cultured.

Hereinafter, the present invention will be described in detail by means of examples.

### EXAMPLE

### Example 1. Isolation and Cryopreservation of Spleen Dendritic Cells (DCs)

Mouse spleen DCs were obtained by introducing EDTA-BSS (balanced salt solution) buffer, collagenase, and DNAse tissue-dissolved solution in a spleen of C57BL/6 mouse (Orient); finely cutting the spleen with operating scissors; adding 6 ml of collagenase-treated solution to the product, followed by culturing for 30 minutes. The separated entire spleen tissues were filtered using 70 μM cell strainer (Falcon) and then centrifuged at 1800 rpm for 5 minutes so as to separate cells. A red blood cell dissolving buffer (Sigma Aldrich) was added to remove red blood cells, and then washed with RPMI-1640 medium containing fetal bovine serum (FBS, Sigma Aldrich) twice. The dendritic cells (DCs) were reacted using microbeads (Miltenyi Biotec) combined with CD11c⁺ antibody, followed by isolation using CD11c Microbeads Ultrapure (MACS, Miltenyi Biotec). 1.2×10⁸ cells were obtained from the spleen tissues, and 7.5×10⁶ DCs were also isolated. 3×10⁶ primary DCs isolated from the spleen were included in RPMI-1640 culture medium containing 10% DMSO, and FBS with different concentrations was added thereto to prepare a vial containing 1 mL solution, followed by freezing and storage in a nitrogen tank for 1 week. The cryopreserved cells were thawed in a shaking incubator at 37° C. and then used.

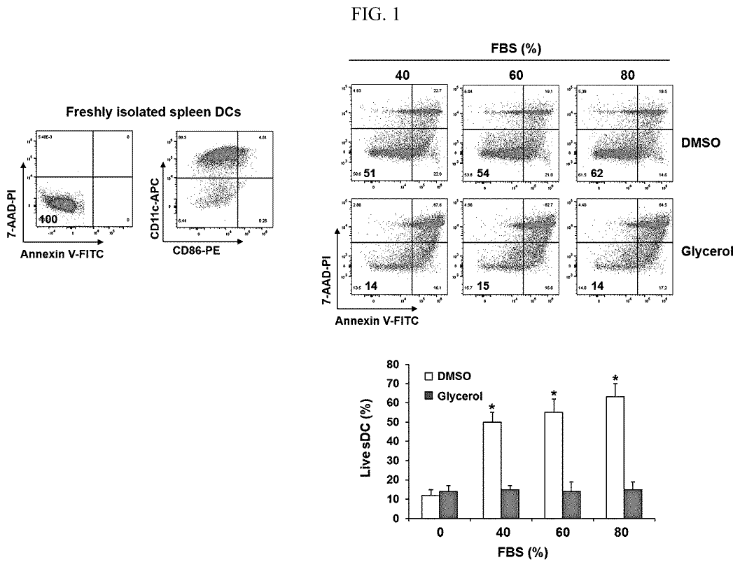

Referring to FIG. 1, survival and death of spleen dendritic cells (sDCs) after thawing was determined by staining the cells with 7-aminoactinomycin D (7-AAD) and annexin V-FITC, using a flow cytometer (MACSQuant VYB flow cytometer, Miltenyi Biotec.). The results measured using the flow cytometer was analyzed using FlowJo v10 (Tree Star, Ashland). If not adding FBS to the cryopreservation agent, it exhibited the cell viability of 10% or less. However, when the concentration of FBS was increased, the cell viability was also increased. For example, when 80% FBS was added, it showed the viability of 62%. * denotes P<0.05 as compared to the control group (0% FBS), which shows statistical significance (n=3).

### Example 2. Measurement of Activation of DCs Using Flow Cytometer

In order to determine whether spontaneous activation occurs when primary DCs isolated from the spleen were cultured, an expression level of CD86 on the surface of primary DCs was analyzed by a flow cytometer. The CD86 expression level was indicated by percentage of CD11c-stained cells using PE-combined CD86 antibody (CD86-PE).

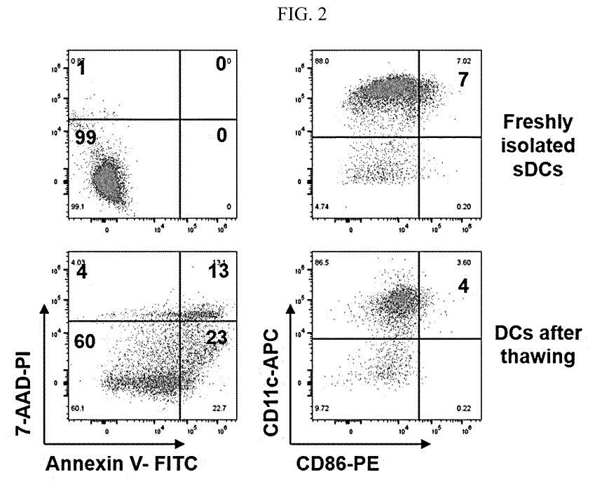

Referring to FIG. 2, DCs immediately after isolation from the spleen (in a state of not being stimulated) have a very low expression of CD86 as a co-stimulating molecule. In Example 1, the DCs, which were cryopreserved in the composition including 10% DMSO and 80% FBS and then thawed, exhibited low CD86 expression level of 4% on the surface of cells.

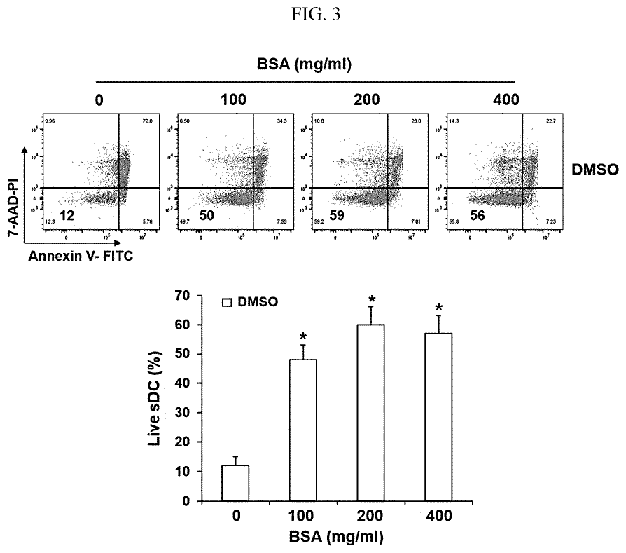

Referring to FIG. 3, DCs prepared by dispensing primary spleen dendritic cells (sDCs) isolated from the spleen in a composition containing 10% DMSO and BSA added at different concentrations, followed by cryopreservation in a nitrogen tank for 7 days and thawing the same, were subjected to measurement of cell viability. In the case of adding 100 mg/mL of BSA, the cell viability was remarkably increased. * denotes P<0.05 as compared to the control group (0 mg/mL BSA), which shows statistical significance (n=3).

### Example 3. Measurement of Viability of DCs After Culture

After adding fibronectin at a concentration of 200 μg/mL (Sigma Aldrich) to RPMI-1640 medium containing 10% FBS, CD11c⁺ primary DCs isolated from the spleen were dispensed in an amount of 2×10⁵/mL, followed by culturing for 24 hours. Thereafter, cell viability and CD86 expression level were measured by a flow cytometer.

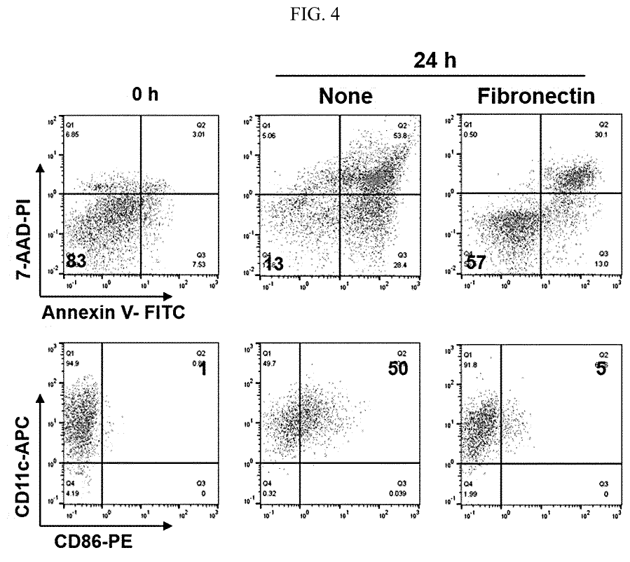

As shown in FIG. 4, in the case where 10% FBS and 200 μg/mL of fibronectin were added to RPMI-1640 medium, it was found that the cell viability was highly maintained to about 57% while an increase in CD86 expression was also inhibited (that is, maintained in non-activated state). On the other hand, when 10% FBS and 200 μg/mL of fibronectin were not used, only 13% cells were survived while activation of cells was greatly increased.

### Example 4. Measurement of Viability of Spleen DCs Thawed After Cryopreservation

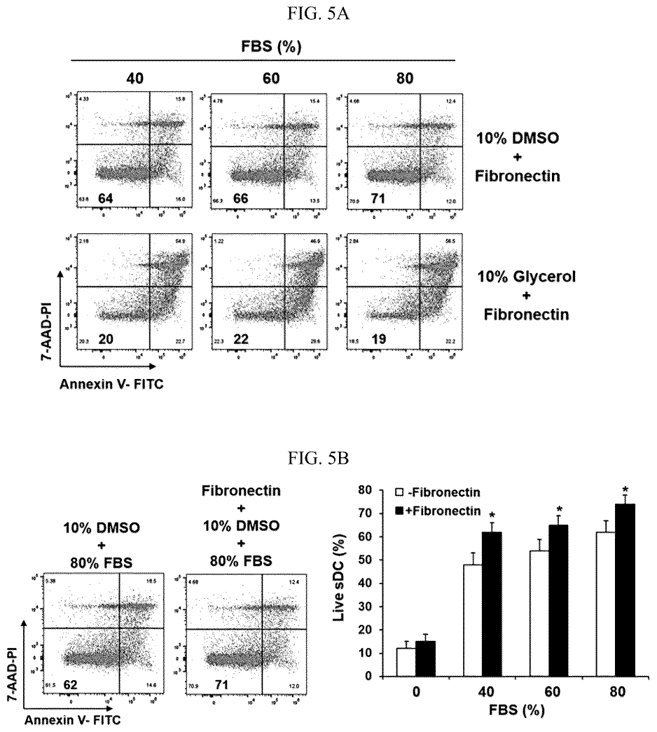

For cells obtained by freezing spleen dendritic cells (sDCs) in a composition for cryopreservation which includes FBS at a concentration of 0%, 40%, 60% or 80%, 10% DMSO and 200 μg/mL of fibronectin for 7 days, and then thawing the same, the viability was measured by a flow cytometer. As a result, as shown in FIG. 5A, the results demonstrated that, if 40%, 60% or 80% FBS is included, the cell viability is highly maintained to 64%, 66% and 71%, respectively.

However, even when the composition for cryopreservation includes FBS at a concentration of 0%, 40%, 60% or 80%, as well as fibronectin, if 10% DMSO is not included (that is, if 10% glycerol is included instead of 10% DMSO), as shown in FIG. 5A, it was confirmed that the cell viability is sharply reduced. Therefore, it could be seen that the composition for cryopreservation should include 10% DMSO.

Further, in the case where fibronectin at a concentration of 200 μg/mL is added to the composition for cryopreservation including 80% FBS and 10% DMSO, as shown in FIG. 5B, it was confirmed that the viability is increased from 62% to 71% by 9% (about 15% based on 62%) due to addition of fibronectin. Therefore, it could be seen that fibronectin has significant influence on cell viability at thawing after cryopreservation. * denotes P<0.05 as compared to the control group (fibronectin), which shows statistical significance (n=3).

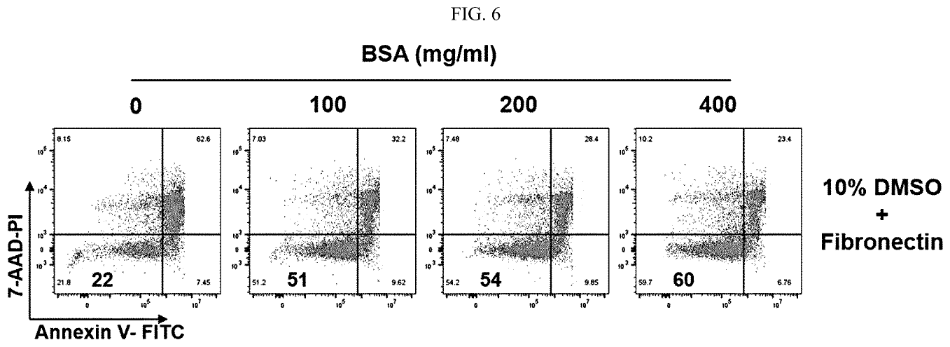

For the composition for cryopreservation including FBS at a concentration of 0%, 40%, 60% or 80%, 10% DMSO and 200 μg/mL of fibronectin, if adding bovine serum albumin (BSA) in an amount of 100 mg/mL, 200 mg/mL or 400 mg/mL instead of FBS, it was confirmed that the viability of primary DCs thawed after cryopreservation ranges from 51 to 60% (FIG. 6).

### Example 5. Measurement of Viability of DCs Isolated After Cryopreservation and Thawing of Total Splenocytes

A composition for cryopreservation, including RPMI-1640 medium (including RPMI-1640, 1 mM sodium pyruvate, 2% penicillin/streptomycin, 100 μM β-mercaptoethanol, 5×HEPES buffer, and 0.2% sodium bicarbonate), 10% DMSO or 10% glycerol, and 0%, 20%, 40%, 60% or 80% FBS was prepared.

To the above composition for cryopreservation, total splenocytes isolated from the spleen were dispensed at a density of 3×10⁷/mL, and the total splenocytes were frozen for 7 days and then thawed to obtain total cells. Then, primary DCs were obtained from the total cells using CD11c microbeads. The survival rate of DCs was measured by a flow cytometer.

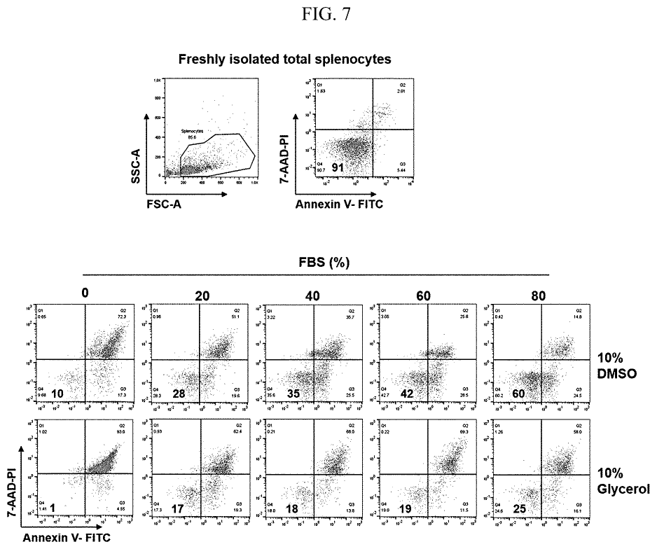

As shown in FIG. 7, when the composition for cryopreservation including 10% DMSO was used, the viability of spleen DCs was increased up to 60% with an increase in FBS concentration. On the other hand, using the composition for cryopreservation including 10% glycerol resulted in maximum 25% viability of spleen DCs.

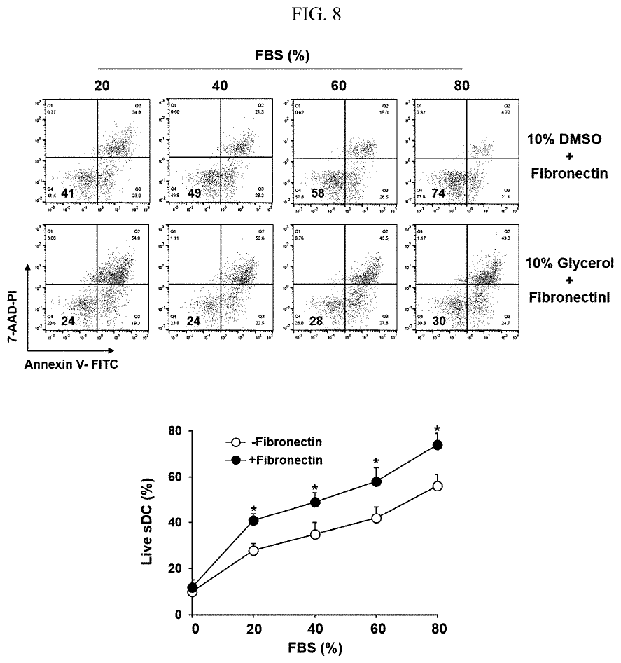

Total splenocytes in a composition for cryopreservation prepared by adding 200 μg/mL of fibronectin to the above composition for cryopreservation were freezing-stored for 7 days, followed by separating the total cells and obtaining spleen primary DCs (sDCs) therefrom. The DCs showed maximum 74% viability (FIG. 8). This demonstrates a result of 14% increase as compared to the case without addition of fibronectin (60% in FIG. 7). * denotes P<0.05 as compared to the control group (fibronectin), which shows statistical significance (n=3).

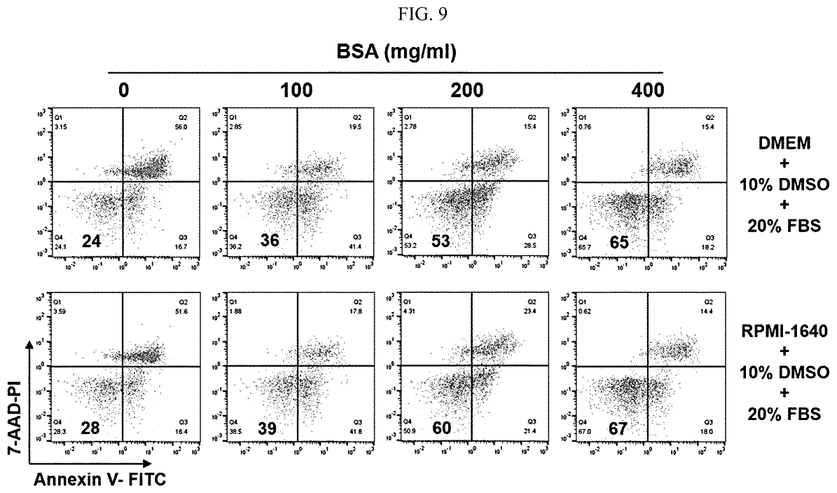

Referring to FIG. 9, the compositions for cryopreservation were prepared by adding 10% DMSO, 20% FBS and BSA with different concentrations to RPMI-1640 medium or DMEM medium. Total splenocytes isolated from the spleen were dispensed at a density of 1×10⁸/mL, followed by freezing the total splenocytes for 7 days and then thawing the same to obtain total cells. Primary DCs were obtained from the total cells and subjected to measurement of survival rate of the DCs by a flow cytometer. As shown in FIG. 9, two types of media demonstrated similar results that the cell viability is increased with an increase in FBS concentration. For example, when 400 mg/mL BSA was added, the viability of splenocytes was 65% and 67%, respectively.

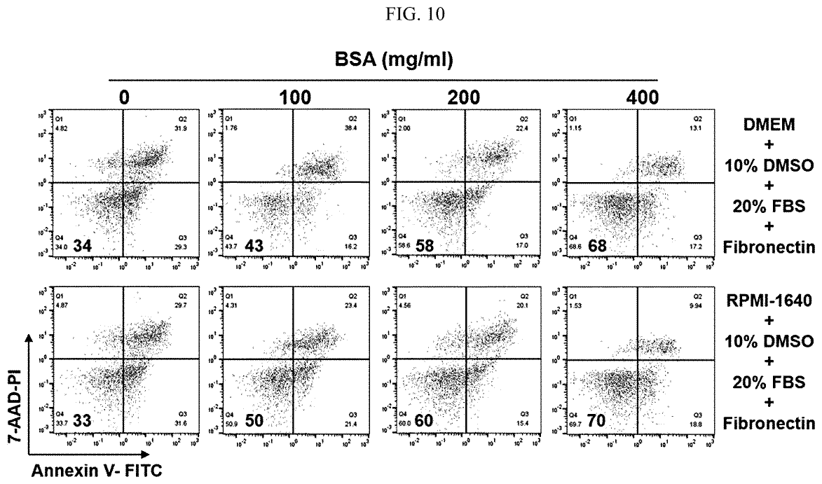

When 200 μg/mL of fibronectin was added to the composition for cryopreservation in FIG. 9 and the viability of primary DCs was measured by the same method, the viability was not greatly increased by addition of fibronectin (FIG. 10).

### Example 6. Measurement of Viability of Spleen DCs Co-Cultured With Fibroblasts

### (1) Preparation of Cell Scaffold

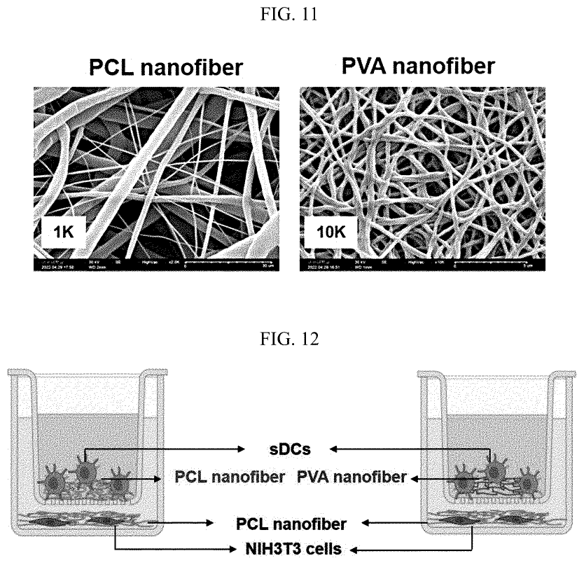

Polycaprolactone (PCL) nanofiber and polyvinyl alcohol (PVA) nanofiber to be used as a scaffold were fabricated in order to co-culture spleen DCs of the present invention and fibroblasts (FIG. 11).

The polycaprolactone (PCL) nanofiber was produced by the method described in Korean Patent Registration No. 10-1684698. More particularly, polycaprolactone (PCL, Mw=80,000, Sigma-Aldrich) was dissolved in chloroform (Sigma Aldrich) at a concentration of 15%. PCL solution (5 mL) was spun at a spinning rate of 8 μl/min with a spinning distance of 20 cm by an electric spinner (NanoNC Co.) using a 25 G metal syringe at 10 KV thus to produce the nanofiber.

The polyvinyl alcohol (PVA) nanofiber was also produced by the method described in the above Korean Patent Registration No. 10-1684698. More particularly, polyvinylalchol (PVA, Mw=89,000-98,000, Sigma-Aldrich) and polyacrylic acid (PAA, Mw=2000, Sigma-Aldrich) were dissolved at 85° C. for 24 hours at a concentrations of 10% (w/v) and 0.2% (w/v), respectively. Glutaraldehyde (GA, 2%, Sigma-Aldrich) was added to the dissolved PVA solution and then mixed for 30 minutes to prepare a solution for electric spinning. 4 mL of the electric spinning solution was spun at a spinning rate of 8 ul/min with a spinning distance of 10 cm using two metal syringes at 10 KV. PVA/PAA/GA nanofibers produced through electric spinning were heated at 60° C. for 1 minute. In order to cross-link the nanofibers, the nanofibers were treated in hydrochloric acid vapor for 60 seconds. Then, PVA nanofiber membrane was immersed in a culture medium for 12 hours or more before use, followed by exposing the same to UV rays for 12 hours to perform sterilization.

### (2) Co-Culture of Spleen DCs and Fibroblasts

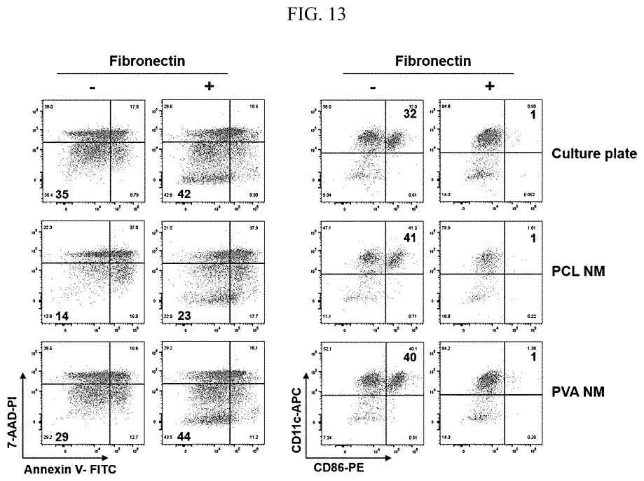

As shown in FIG. 12, in order to co-culture two types of cells, a culture system was fabricated by adhering PCL nanofiber membrane and PVA nanofiber membrane on the insert in an upper layer of trans-well and adhering PCL nanofiber membrane on the base in a lower layer of the trans-well. On a lower layer of the culture system, NIH3T3 cells as the fibroblasts (1×10⁶/mL) were dispensed and cultured for 3 days, followed by dispensing the DCs thawed after cryopreservation (2×10⁵/mL) in the upper layer. Immediately afterwards, two layers were fabricated and then cultured for 24 hours. The DCs used herein were obtained after cryopreservation the cells in the composition including 10% DMSO, 80% FBS and 200 μg/mL of fibronectin (viability of 60%).

Further, a typical culture plate was prepared and the cryopreserved, and then thawed the DCs (2×10⁵/mL) and the fibroblasts, that is, NIH3T3 cells (1×10⁶/mL) were dispensed thereon and cultured by the same method as described above.

As shown in FIG. 13, when the spleen DCs were dispensed at a density of 2×10⁵/mL in a culture plate including RPMI-1640 medium (not containing fibronectin) and cultured for 24 hours, it was found that the viability of DCs was 35% while spontaneously activated cells showed 32% viability. In the case where fibronectin was added at a concentration of 200 μg/mL to the culture medium, the viability of DCs was a little increased to 42% but the spontaneously activated cells were considerably suppressed to 1%. When PCL nanofiber and PVA nanofiber were adhered to a culture plate and spleen DCs were cultured thereon, the viability and activation inhibiting effects were generally similar to those in the case of culturing spleen DCs on the culture plate. This result demonstrates that the spleen DCs of the present invention may be cultured not only in the plate as a two-dimensional culture but also on a nanofiber scaffold as a three-dimensional culture.

### Example 7. Measurement of Cell Viability After Culture of DCs Isolated From Cryopreserved Total Splenocytes

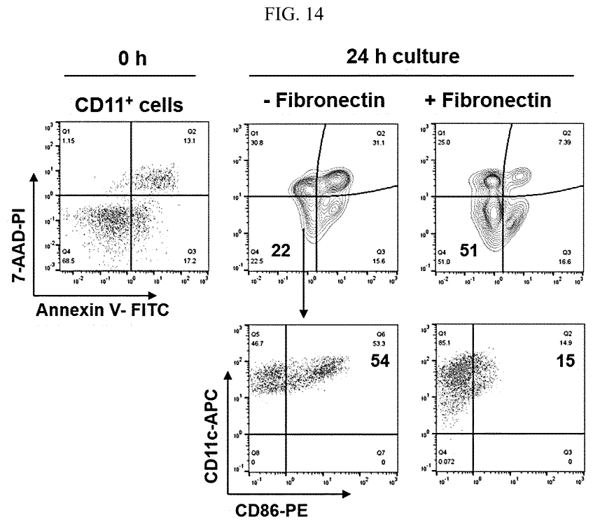

Referring to FIG. 14, total splenocytes were cryopreserved at 3×10⁷/mL in the composition (1 mL) containing 10% DMSO, 80% FBS and 200 μg/mL of fibronectin and thawed after 7 days, followed by separating 1×10⁶ CD11c⁺ spleen DCs using CD11c antibody-attached microbeads. When the DCs (2×10⁵ cells) isolated from the cryopreserved total splenocytes were dispensed on the culture plate and cultured in RPMI-1640 medium for 24 hours, the cell viability was very low to about 22%, and 54% of survived cells showed CD86 expression. When fibronectin was added in a condition that the cells were cultured in the culture plate, the cell viability was significantly increased while spontaneous activation was considerably inhibited.

### Example 8. Measurement of Viability of DCs After Co-Culture of Cryopreserved DCs and Fibroblasts

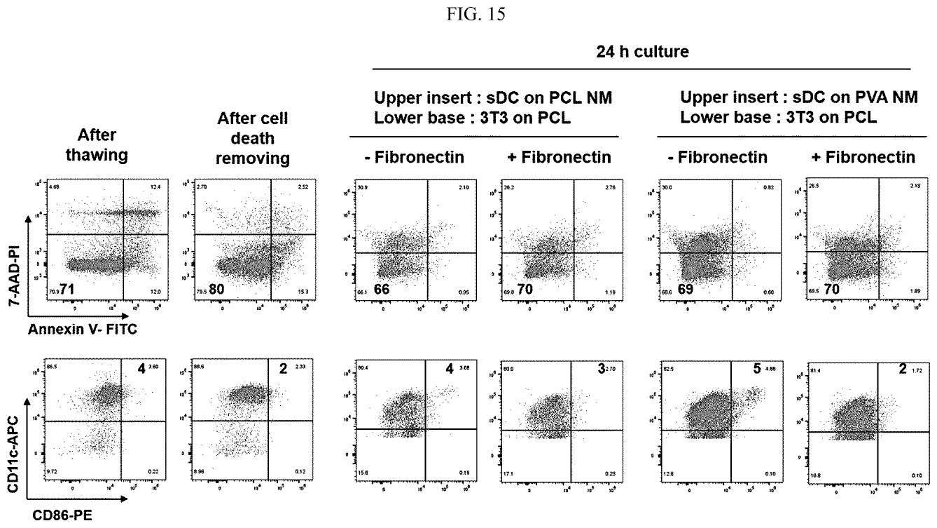

Referring to FIG. 15, CD11c⁺ DCs isolated from the spleen were cryopreserved at 1×10⁶/mL in the composition (1 mL) containing 10% DMSO, 80% FBS and 200 μg/mL of fibronectin and thawed after 7 days. The viability of the thawed DCs was confirmed, and dead cells were removed using dead cell removal microbeads (Miltenyi Biotec) while 2×10⁵ survived cells only were dispensed on the upper PCL or PVA nanofiber membrane layer fabricated for co-culture. On the other hand, on the lower PCL nanofiber membrane layer for co-culture, 1×10⁶ NIH3T3 fibroblasts were dispensed and cultured for 3 days, followed by combining the same with the upper membrane layer on which the DCs were dispensed, and then, co-culturing the same for 24 hours. When the DCs were dispensed on the upper PCL or PVA nanofiber while the fibroblasts were co-cultured on the base layer, the cell viability was remarkably increased while the DCs were maintained in non-active.

When the cryopreserved DCs were co-cultured, the cell viability was not greatly increased due to addition of fibronectin. This result proposes that survival and activity of the primary dendritic cells (DCs) may be stabilized by secretion of extracellular matrix components such as fibronectin or other unknown soluble factors from the co-cultured fibroblasts.

1-18. (canceled)

19. A method for culturing primary dendritic cells (DCs), the method comprising:
freezing and storing the primary DCs in a composition comprising serum, dimethyl sulfoxide (DMSO) and at least one of fibronectin and albumin;
thawing the primary DCs; and
after thawing, culturing the primary DCs.

20. The method of claim 19, wherein the primary DCs are included in tissues.

21. The method of claim 19, wherein the composition includes the serum, the dimethyl sulfoxide (DMSO) and the fibronectin.

22. The method of claim 19, wherein the culturing comprises culturing the primary DCs in a medium including fibronectin.

23. The method of claim 19, wherein the culturing comprises culturing the primary DCs in a medium which does not include fibronectin.

24. The method of claim 19, wherein the culturing comprises co-culturing the primary DCs together with fibroblast.

25. The method of claim 19, wherein the culturing comprises:
dispensing the primary DCs or the tissues including the primary DCs on a first scaffold which includes polyvinyl alcohol (PVA) nanofiber or polycaprolactone (PCL) nanofiber; and
dispensing fibroblasts on a second scaffold which includes polycaprolactone (PCL) nanofiber thus to co-culture the primary DCs and the fibroblasts.

26. The method of claim 25, wherein the first scaffold and the second scaffold are laminated in an incubator while being spaced apart from each other.

27. The method of claim 19, wherein the serum is fetal bovine serum (FBS), and a concentration of the FBS is in the range of 10 to 90% (v/v) based on a total volume of the composition.

28. The method of claim 19, wherein a concentration of the DMSO is in the range of 5 to 15% (v/v) based on a total volume of the composition.

29. The method of claim 21, wherein the fibronectin is included in an amount of 100 to 300 μg/mL.

30. The method of claim 19, wherein the composition includes the serum, the dimethyl sulfoxide (DMSO) and the albumin.

31. The method of claim 30, wherein the albumin is included in an amount of 300 to 500 mg/mL.

32. The method of claim 19, wherein the composition includes the serum, the dimethyl sulfoxide (DMSO), the fibronectin and the albumin.

33. A composition for cryopreservation of primary DCs, the composition comprising serum, dimethyl sulfoxide (DMSO) and at least one of fibronectin and albumin.

34. The composition of claim 33, wherein the composition comprises the serum, the dimethyl sulfoxide (DMSO) and the fibronectin.

35. The composition of claim 34, wherein the composition comprises the serum, the dimethyl sulfoxide (DMSO) and the albumin.

36. The composition of claim 33, wherein the serum is 10 to 90% FBS, and the DMSO is 5 to 15% DMSO.

37. The composition of claim 33, wherein the fibronectin is included in an amount of 100 to 300 μg/mL.

38. The composition of claim 33, wherein the albumin is included in an amount of 300 to 500 mg/mL.
